(19) United States
(12) Patent Application Publication
(10) Pub. No.: US 2014/0170085 A1
(43) Pub. Date: Jun. 19, 2014

(54) COSMETIC PREPARATION TAILORED TO THE INDIVIDUAL USER AND PROCESS FOR PRODUCING SAME

(71) Applicant: BEIERSDORF AG, Hamburg (DE)

(72) Inventors: Ann-Sophie PETERS, Hamburg (DE); Heiner Gers-Barlag, Kummerfeld (DE)

(73) Assignee: Beiersdorf AG, Hamburg (DE)

(21) Appl. No.: 13/745,433

(22) Filed: Jan. 18, 2013

(30) Foreign Application Priority Data
Dec. 18, 2012 (DE) 102012223340.7

(51) Int. Cl. A61K 8/35; A61Q 19/00; A61K 8/67

(52) U.S. Cl. CPC A61K 8/355; A61K 8/678; A61Q 19/00
USPC 424/59; 424/94.1; 514/690; 53/428

(57) ABSTRACT

A process for producing a cosmetic preparation wherein a cosmetic base, admixed with one or more cosmetic partial preparations or active ingredient concentrates, is mounted, in the package intended for the end user, in a dually asymmetric centrifuge, and is centrifuged in such a way that the cosmetic part preparations or active ingredient concentrates are distributed uniformly in the cosmetic base.

### CROSS-REFERENCE TO RELATED APPLICATIONS

The present application claims priority under 35 U.S.C. §119 of German Patent Application No. 10 2012 223 340.7, filed Dec. 18, 2012, the entire disclosure of which is expressly incorporated by reference herein.

### BACKGROUND OF THE INVENTION

1. Field of the Invention

The present invention relates to a process for producing a cosmetic preparation that is tailored to the individual user.

2. Discussion of Background Information

The desire to look beautiful and attractive is naturally rooted in people. Even though the beauty ideal has undergone changes over the course of time, the desire for a flawless outward appearance has always been the aim of mankind. A significant part of a beautiful and attractive appearance is the condition and the appearance of the skin.

In order that the skin can completely fulfill its biological functions, it requires regular cleaning and care. Cleaning the skin serves here to remove dirt, sweat and remains of dead skin particles, which form an ideal breeding ground for pathogens and parasites of all types. Skin care products mostly serve to moisturize and regrease the skin. Active ingredients are often added to them which are intended to regenerate the skin and, for example, prevent and reduce its premature aging (e.g. the formation of wrinkles, lines).

However, one disadvantage in the prior art is the fact that conventional cosmetic preparations comprise already stipulated concentrations and combinations of active ingredients, for example UV filters or skin moisturizers, which are not matched to the individual requirements of the individual user. However, since skin types and application areas of cosmetics are as diverse as the characters of people, there is a great need for cosmetics that are tailored individually to the requirements of the individual consumer.

Consumers wanting such an “individual cosmetic” currently have to rely on concocting these themselves in a domestic kitchen using the “building block principle”. However, this has the disadvantage that such processes take place in mostly questionable hygienic circumstances and with inadequate homogenization on account of the production technology, as a result of which not just the shelf life of the products produced in this way suffers. Moreover, with prior methods, the ratio of raw materials used to the end product is extremely unfavorable because a large number of pots, stirrers and other equipment come into contact with the starting materials and the end product and—unavoidably—corresponding remnants are left sticking to these. This leads not only to a disproportionately high raw material consumption, but moreover to time-consuming and environmentally-impacting cleaning procedures following cosmetic production.

Also known is the use of so-called double-chamber products in which two (or more) individual components of a cosmetic are located in separate chambers of a product dispenser and are removed from the storage vessel via one or more openings or via a common mixing nozzle. With these products, although the stated hygiene problems no longer arise, what does arise here as well is the “wastage” of partial preparations since the individual preparations are not used up to the same degree and in the event of individual use of the partial preparations, relatively large amounts of leftovers of individual components are almost inevitable.

It would therefore be advantageous to have available a process for producing individually adjusted cosmetics which can be carried out quickly and in a resource-conserving and perfectly hygienic manner.

### SUMMARY OF THE INVENTION

The present invention provides process for producing a cosmetic preparation, which process comprises combining a cosmetic base with one or more cosmetic partial preparations and/or active ingredient concentrates, and thereafter centrifuging it in a package intended for an end user in a dually asymmetric centrifuge to distribute the one or more cosmetic partial preparations and/or active ingredient concentrates uniformly within the cosmetic base.

In one aspect of the process, the cosmetic base may be an emulsion, e.g., an O/W emulsion.

In another aspect, the cosmetic base may have a viscosity of from 3000 to 40,000 mPas and/or the one or more cosmetic partial preparations or active ingredient concentrates may have a viscosity of from 1 to 20,000 mPas, or may be present in the form of powders.

In yet another aspect of the process, the one or more cosmetic partial preparations and/or active ingredient concentrates may comprise one or more active ingredients selected from glycyrrhetic acid, urea, arctiin, alpha-lipoic acid, folic acid, phytoene, D-biotin, coenzyme Q10, alpha-glucosylrutin, carnitine, carnosine, caffeine, natural and/or synthetic isoflavonoids, glycerylglucose, creatine, creatinine, taurine, tocopherol, tocopherol acetate, β-alanine, bisabolol, vitamin C, panthenol, and licochalcone A.

In a still further aspect, the one or more cosmetic partial preparations and/or active ingredient concentrates may comprise one or more UV filters and/or may comprise one or more active ingredients selected from dyes and effect pigments approved for cosmetics.

In another aspect, the cosmetic base may comprise one or more 0/W emulsifiers selected from glyceryl stearate citrate, glyceryl stearate (self-emulsifying), stearic acid, stearate salts, polyglyceryl-3 methylglycose distearate, ceteareth-20, PEG-40 stearate, and sodium cetearyl sulfate and/or may comprise cetearyl alcohol in combination with at least one of PEG-40 hydrogenated castor oil, sodium cetearyl sulfate, glyceryl stearate, and potassium cetyl phosphate.

In another aspect, the cosmetic base may comprise one or more W/O emulsifiers selected from polyglyceryl-2 dipolyhydroxystearate, PEG-30 dipolyhydroxystearate, cetyldimethicone copolyol, and polyglyceryl-3 diisostearate.

In another aspect, the cosmetic base may comprise one or more thickeners, e.g., one or more thickeners selected from polyacrylates (carbopols), acrylates/C10-30 alkyl acrylate crosspolymers, ammonium acryloyldimethyltaurate/VP copolymers, starches, starch derivatives, xanthan gum, and guar derivatives.

In another aspect, the one or more cosmetic partial preparations and/or active ingredient concentrates may comprise one or more perfume oils, or may be used for perfuming.

In another aspect, the package intended for the end user may comprise or be made of one or more of polyethylene, polypropylene, polyethylene terephthalate, and glass.

In another aspect, the volume ratio of cosmetic base to total amount of cosmetic partial preparations and active ingredient concentrates may be from 1:10 to 1000:1.

In another aspect, a centrifuge rotational speed of from 300 to 3500 rpm may be used and/or centrifuging may be carried out for a period of from 5 to 300 seconds.

In another aspect, the amount of cosmetic preparation which is prepared in one process cycle is from 5 to 250 g and/or the package may have a volume of from 30 to 100 ml.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description making apparent to those skilled in the art how the several forms of the present invention may be embodied in practice.

In the process for producing a cosmetic preparation of the present invention a cosmetic base, admixed with one or more cosmetic partial preparations or active ingredient concentrates, is mounted, in the package intended for the end user, in a dually asymmetric centrifuge, and is centrifuged in such a way that the one or more cosmetic partial preparations or active ingredient concentrates are distributed uniformly in the cosmetic base.

Dually asymmetric centrifugation is understood here as meaning a centrifugation in two directions.

This process dispenses with the need for all other customary cleaning steps since no stirrers, stifling beakers and the like are used. Moreover, the otherwise customary “standing times” or “dead times” before repeating the process or changing storage cartridges do not arise. On account of the fact that all body-related or object-related contamination sources for germs, impurities etc. are excluded in the production process, the process is particularly hygienic. Consequently, the content of preservatives in the cosmetic preparation can be kept particularly low, which is a further advantage compared with the prior art.

Although WO 2002/015735, US 2006/0036454, DE 102008023721, DE 4110299, WO 98/030189, WO 2011/155765, EP 1237100 and EP 1674081 exist, these documents, the entire disclosures of which are incorporated by reference herein, were unable to point the way to the present invention.

Within the context of the present disclosure, the characterizations as “according to the invention”, “advantageous according to the invention” etc. apply both to the process according to the invention and also to the process product produced by this process which is likewise in accordance with the invention.

It is advantageous according to the invention if the cosmetic base is an emulsion and preferably an O/W emulsion.

Embodiments of the present invention that are advantageous according to the invention are also those in which the cosmetic base has a viscosity of from about 1000 to about 40,000 mPas. According to the invention, the range from 5000 to 40,000 mPas is preferred here.

In this connection, it is particularly preferable for face care products if the cosmetic base has a viscosity of from 15,000 to 30,000 mPas. Face care products constitute a particularly preferred embodiment of the preparation according to the invention.

Moreover, it is advantageous according to the invention if the one or more partial preparations or active ingredient concentrates have a viscosity of from about 1 to about 20,000 mPas or are in the form of powders. According to the invention, the range from 1 to 10,000 mPas is preferred.

Embodiments of the present invention that are advantageous according to the invention are ones in which the partial preparations or active ingredient concentrates comprise one or more active ingredients selected from glycyrrhetic acid, urea, arctiin, alpha-lipoic acid, folic acid, phytoene, D-biotin, coenzyme Q10, alpha-glucosylrutin, carnitine, carnosine, caffeine, natural and/or synthetic isoflavonoids, glycerylglucose, creatine, creatinine, taurine, tocopherol, tocopherol acetate, β-alanine, bisabolol, vitamin C, panthenol and/or licochalcone A.

Furthermore, it is advantageous according to the invention if the partial preparations or active ingredient concentrates comprise one or more UV filters and in particular, UV filters selected from 2-phenylbenzimidazole-5-sulfonic acid and/or salts thereof; phenylene-1,4-bis(2-benzimidazyl)-3,3′-5,5′ -tetrasulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl)sulfonic acid salts; 2,2′-methylenebis(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]disiloxanyl]propyl]phenol; 4-bis{[4-(2-ethylhexyloxy)-2-hydroxy]phenyl}-6-(4-methoxyphenyl)-1,3,5 -triazine; 3-(4-methylbenzylidene)camphor; 3-benzylidenecamphor; ethylhexyl salicylate; terephthalidenedicamphorsulfonic acid; 4-(tert-butyl)-4′ -methoxydibenzoyl-methane; 2-ethylhexyl 2-cyano-3,3-diphenylacrylate; 2-ethylhexyl 4-(dimethylamino)benzoate; amyl 4-(dimethylamino)benzoate; di(2-ethylhexyl) 4-methoxybenzalmalonate; 2-ethylhexyl 4-methoxycinnimate; isoamyl 4-methoxycinnimate; 2-hydroxy-4-methoxybenzophenone, 2-hydroxy-4-methoxy-4′-methylbenzophenone; 2,2′-dihydroxy-4-methoxybenzophenone; hexyl 2-(4′-diethylamino-2′-hydroxybenzoyl)benzoate; homomenthyl salicylate; 2-ethylhexyl 2-hydroxybenzoate; dimethicodiethyl benzalmalonate; 3-(4-(2,2-bisethoxycarbonylvinyl)phenoxy)-propenyl)methoxysiloxane/dimethylsiloxane copolymer; dioctylbutylamidotriazone (INCI: Diethylhexyl-Butamidotriazone); 2,4-bis [5-1 (dimethylpropyl)benzoxazol-2-yl-(4-phenyl)imino]-6-(2-ethylhexyl)imino-1,3,5-triazine with the (CAS No. 288254-16-0); tris(2-ethylhexyl) 4,4′,4″-(1,3,5-triazine-2,4,6-triyltriimino)trisbenzoate (also: 2,4,6-tris [anilino(p-carbo-2′-ethyl-1′-hexyloxy)]-1,3,5-triazine (INCI: Ethylhexyl Triazone); 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine); 2,4,6-tribiphenyl-4-yl-1,3,5-triazine; merocyanine; titanium dioxide; and zinc oxide.

In an alternative embodiment of the present invention, the UV filter or filters are present in the cosmetic base.

According to the invention, it further is advantageous if the partial preparations comprise perfume substances. In an alternative embodiment of the present invention, the perfume substances are present in the cosmetic base.

Last but not least, it is advantageous according to the invention if the partial preparations or active ingredient concentrates comprises one or more active ingredients selected from dyes and effect pigments approved for cosmetics.

If the cosmetic base is in the form of an O/W emulsion (oil-in-water emulsion), it is advantageous according to the invention if the cosmetic base comprises one or more O/W emulsifiers, in particular O/W emulsifiers selected from glyceryl stearate citrate, glyceryl stearate (self-emulsifying), stearic acid, stearate salts, polyglyceryl-3 methylglycose distearate, ceteareth-20, PEG-40 stearate, sodium cetearyl sulfate. It is also advantageous within the context of the present invention to use cetearyl alcohol in combination with one or more PEG-40 hydrogenated castor oil, sodium cetearyl sulfate and glyceryl stearate, and potassium cetyl phosphate.

If, by contrast, the cosmetic base is in the form of a W/O emulsion (water-in-oil emulsion), the embodiments advantageous according to the invention are ones wherein the cosmetic base comprises one or more W/O emulsifiers and in particular, W/O emulsifiers selected from polyglyceryl-2 dipolyhydroxystearate, PEG-30 dipolyhydroxystearate, cetyl dimethicone copolyol, and polyglyceryl-3 diisostearate.

However, the cosmetic base can also be present as an emulsifier-free preparation e.g. as hydrodispersion.

It is advantageous according to the invention if the cosmetic base comprises one or more thickeners such as, e.g., thickeners selected from polyacrylates (carbopols), acrylates/C10-30 alkyl acrylate cross polymers, ammonium acryloyldimethyltaurate/VP copolymers, starches, starch derivatives, xanthan gum, and guar derivatives.

The partial preparations according to the invention may also comprise perfume oils, and/or may be used for perfuming.

Overall, embodiments that are advantageous according to the invention are obtained if the volume ratio of cosmetic base to total amount of cosmetic partial preparations and active ingredient concentrates is from about 1:10 to about 1000:1, preference being given to the range of from 1:1 to 1000:1.

As regards the package intended for the end user, the embodiments advantageous according to the invention are ones wherein the package intended for the end user comprises or is made of one or more plastic materials such as polyethylene, polypropylene, polyethylene terephthalate, or is made of or comprises glass.

The process according to the invention results in products that are particularly advantageous if the package intended for the end user (and thus the sum of cosmetic base and cosmetic partial preparations and also active ingredient concentrates in the end product) has a volume of not more than about 30 ml. For example, the volume may be from about 30 to about 100 ml, preferably from 30 to 50 ml.

It further is advantageous if the amount of cosmetic preparation which is produced in one process cycle is not higher than about 250 g, e.g. not higher than about 200 g, not higher than about 150 g, or not higher than about 100 g, but usually not lower than about 5 g, e.g., not lower than about 10 g, not lower than about 25 g, or not lower than about 50 g. For example, the preferred amount is from 50 to 100 g.

It is advantageous according to the invention if the mixing process is carried out at a centrifuge rotational speed of not less than about 300 rpm and not higher than about about 3500 rpm. Preferably, up to about 450 g will act on the material being mixed at full speed.

In this regard, it further is advantageous for the large axis to rotate with a counter movement relative to the small axis. Usually, the axes rotate in a ratio of from about 1:10 to about 10:1. Preference is given to a ratio of from 1:4 to 4:1. As a result, the ratio of centrifugal force to shear force is adjusted.

It is also advantageous if the mixing time is from about 5 to about 300 seconds, a mixing time of from 10 to 120 seconds being preferred.

According to the invention, preference is given for example to using the “SpeedMixer”™ DAC 150.1 FVZ from Hauschild Engineering for carrying out the process according to the invention.

Further devices that are advantageous for this purpose are CAVEX Alginate Mixer II from Cavex Holland BV or TurboMAX® Alginate Auto Mixer from DENTSPLY International.

According to the invention, the mixing unit is advantageously one wherein no store of raw material or raw material preparations is present.

It is advantageous according to the invention if an intelligent label, for example a RFID chip or barcode, indicates the appropriate stirring speed or time and this information can be “read” by the mixing device.

Also in accordance with the invention is a business method for offering the process according to the invention in a sales outlet customary for the selling of cosmetics. According to the invention, this process is then preferably used by a specialist advisor following thorough consultation with the individual customer regarding their individual wishes and requirements, i.e., after ascertaining the required active ingredients and use concentrations, a corresponding preparation is produced individually for the customer in the sales outlet. Accordingly, the present invention also provides a method of providing a customer with a cosmetic product tailored to its individual needs and/or wishes. The process comprises making the cosmetic product by the process of the present invention as set forth above (including the various embodiments thereof), preferably following an assessment of the customer's skin (e.g., by a cosmetic specialist and/or on site) and preferably followed by handing the prepared product to the customer, or shipping the product to the customer. It also is advantageous for this purpose to have available several cosmetic bases which are already adapted for certain skin types such as, e.g., dry skin, normal skin and fatty skin and to which one or more partial preparations and/or active ingredient concentrates that take into account a customer's individual needs and/or wishes can be added.

In an alternative that is advantageous according to the invention, moreover, ordering/reordering via an online tool is also possible.

### WORKING EXAMPLE

### Cosmetic Base

| | |
|---|---|
| Water | 76.85% |
| Glycerin | 7.5% |
| Ethylhexyl Cocoate | 5.0% |
| Methylpropanediol | 3.5% |
| Tapioca Starch | 3.0% |
| Cetearyl Alcohol | 2.0% |
| Dimethicone | 0.9% |
| Phenoxyethanol | 0.5% |
| Carbomer | 0.2% |
| Caprylylglycol | 0.25% |
| Sodium Stearoyl Glutamate | 0.2% |
| Acrylates/C10-30 Alkyl Acrylate Crosspolymer | 0.1% |

### Partial Preparation 1

| C12-15 Alkyl Benzoate | q.s. |
|---|---|
| Tocopheryl Acetate | 0.25% |
| Ubiquinone | 0.3% |

### Partial Preparation 2

| Alcohol Denat. | q.s. |
|---|---|
| Salicylic Acid | 5.0% |

40 ml of cosmetic base are poured into the pot with 5 ml of partial preparation 1 and with 5 ml of partial preparation 2. The constituents are mixed for 90 seconds at 2000 rpm in a “SpeedMixer”™ DAC 150.1 FVZ from Hauschild Engineering.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A process for producing a cosmetic preparation, wherein the process comprises combining a cosmetic base with one or more cosmetic partial preparations or active ingredient concentrates, and thereafter centrifuging it in a package intended for an end user in a dually asymmetric centrifuge to distribute the one or more cosmetic partial preparations or active ingredient concentrates uniformly within the cosmetic base.

2. The process of claim 1, wherein the cosmetic base is an emulsion.

3. The process of claim 2, wherein the emulsion is an O/W emulsion.

4. The process of claim 1, wherein the cosmetic base has a viscosity of from 3000 to 40,000 mPas.

5. The process of claim 1, wherein the one or more cosmetic partial preparations or active ingredient concentrates have a viscosity of from 1 to 20,000 mPas or are present in the form of powders.

6. The process of claim 1, wherein the one or more cosmetic partial preparations or active ingredient concentrates comprise one or more active ingredients selected from glycyrrhetic acid, urea, arctiin, alpha-lipoic acid, folic acid, phytoene, D-biotin, coenzyme Q10, alpha-glucosylrutin, carnitine, carnosine, caffeine, natural and/or synthetic isoflavonoids, glycerylglucose, creatine, creatinine, taurine, tocopherol, tocopherol acetate, β-alanine, bisabolol, vitamin C, panthenol, and licochalcone A.

7. The process of claim 1, wherein the one or more cosmetic partial preparations or active ingredient concentrates comprise one or more UV filters selected from 2-phenylbenzimidazole-5-sulfonic acid and salts thereof; phenylene-1,4-bis(2-benzimidazyl)-3,3′-5,5′ -tetrasulfonic acid salts; 1,4-di(2-oxo-10-sulfo-3-bornylidenemethyl)benzene and salts thereof; 4-(2-oxo-3-bornylidenemethyl)benzenesulfonic acid salts; 2-methyl-5-(2-oxo-3-bornylidenemethyl)sulfonic acid salts; 2,2′ -methylenebis(6-(2H-benzotriazol-2-yl)-4-(1,1,3,3-tetramethylbutyl)phenol); 2-(2H-benzotriazol-2-yl)-4-methyl-6-[2-methyl-3-[1,3,3,3-tetramethyl-1-[(trimethylsilyl)oxy]disiloxanyl]propyl]phenol; 4-bis{[4-(2-ethylhexyloxy)-2-hydroxy]phenyl }-6-(4-methoxyphenyl)-1,3,5-triazine; 3-(4-methylbenzylidene)camphor; 3-benzylidenecamphor; ethylhexyl salicylate;
terephthalidenedicamphorsulfonic acid; 4-(tert-butyl)-4′-methoxydibenzoylmethane; 2-ethylhexyl 2-cyano-3,3-diphenylacrylate; 2-ethylhexyl 4-(dimethylamino)benzoate; amyl 4-(dimethylamino)benzoate; di(2-ethylhexyl) 4-methoxybenzalmalonate; 2-ethylhexyl 4-methoxycinnimate; isoamyl 4-methoxycinnimate; 2-hydroxy-4-methoxybenzophenone, 2-hydroxy-4-methoxy-4′-methylbenzophenone; 2,2′-dihydroxy-4-methoxybenzophenone;
hexyl 2-(4′-diethylamino-2′-hydroxybenzoyl)benzoate; homomenthyl salicylate; 2-ethylhexyl 2-hydroxybenzoate; dimethicodiethyl benzalmalonate; 3-(4-(2,2-bisethoxycarbonylvinyl)-phenoxy)propenyl)methoxysiloxane/dimethylsiloxane copolymer; dioctylbutylamidotriazone (INCI: Diethylhexyl-Butamidotriazone); 2,4-bis [5-1(dimethylpropyl)benzoxazol-2-yl-(4-phenyl)imino]-6-(2-ethylhexyl)imino-1,3,5-triazine with the (CAS No. 288254-16-0); tris(2-ethylhexyl) 4,4′,4″-(1,3,5-triazine-2,4,6-triyltriimino)trisbenzoate (also: 2,4,6-tris[anilino(p-carbo-2′-ethyl-1′-hexyloxy)]-1,3,5-triazine (INCI: Ethylhexyl Triazone); 2,4-bis {[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine); 2,4,6-tribiphenyl-4-yl-1,3,5-triazine;
merocyanine; titanium dioxide; and zinc oxide.

8. The process of claim 1, wherein the one or more cosmetic partial preparations or active ingredient concentrates comprise one or more active ingredients selected from dyes and effect pigments approved for cosmetics.

9. The process of claim 1, wherein the cosmetic base comprises one or more O/W emulsifiers selected from glyceryl stearate citrate, glyceryl stearate (self-emulsifying), stearic acid, stearate salts, polyglyceryl-3 methylglycose distearate, ceteareth-20, PEG-40 stearate, and sodium cetearyl sulfate.

10. The process of claim 1, wherein the cosmetic base comprises cetearyl alcohol in combination with at least one of PEG-40 hydrogenated castor oil, sodium cetearyl sulfate, glyceryl stearate, and potassium cetyl phosphate.

11. The process of claim 1, wherein the cosmetic base comprises one or more W/O emulsifiers selected from polyglyceryl-2 dipolyhydroxystearate, PEG-30 dipolyhydroxystearate, cetyldimethicone copolyol, and polyglyceryl-3 diisostearate.

12. The process of claim 1, wherein the cosmetic base comprises one or more thickeners selected from polyacrylates (carbopols), acrylates/C10-30 alkyl acrylate crosspolymers, ammonium acryloyldimethyltaurate/VP copolymers, starches, starch derivatives, xanthan gum, and guar derivatives.

13. The process of claim 1, wherein the one or more cosmetic partial preparations or active ingredient concentrates comprise perfume oils, or are used for perfuming.

14. The process of claim 1, wherein the package intended for an end user comprises polyethylene, polypropylene, polyethylene terephthalate, or glass.

15. The process of claim 1, wherein a volume ratio of cosmetic base to total amount of cosmetic partial preparations and active ingredient concentrates is from 1:10 to 1000:1.

16. The process of claim 1, wherein a centrifuge rotational speed of from 300 to 3500 rpm is used.

17. The process of claim 1, wherein centrifuging is carried out for a period of from 5 to 300 seconds.

18. The process of claim 1, wherein an amount of cosmetic preparation which is prepared in one process cycle is from 5 to 250 g.

19. The process of claim 1, wherein the package has a volume of from 30 to 100 ml.

20. A cosmetic end user product that is prepared by the process of claim 1.
